(19) United States
(12) Patent Application Publication
(10) Pub. No.: US 2025/0333466 A1
(43) Pub. Date: Oct. 30, 2025

(54) COMPOSITIONS AND METHODS FOR TREATMENT OF INFERTILITY IN MALES

(71) Applicant: FERRING B.V., Hoofddorp (NL)

(72) Inventors: Patrick HEISER, Parsippany, NJ (US); Joan-Carles ARCE, Dragoer (DK)

(73) Assignee: FERRING B.V., Hoofddorp (NL)

(21) Appl. No.: 18/868,575

(22) PCT Filed: May 26, 2023
(86) PCT No.: PCT/EP2023/064178

§ 371 Date: Nov. 22, 2024

Related U.S. Application Data
(60) Provisional application No. 63/346,153, filed on May 26, 2022.

(30) Foreign Application Priority Data
Jun. 16, 2022 (EP) 22179438.1

(51) Int. Cl. C07K 14/59; A61K 38/00

(52) U.S. Cl. CPC C07K 14/59; A61K 38/00

(57) ABSTRACT

Methods, uses and compositions including rFSH including α2,3- and α2,6-sialylation for use in the treatment of infertility in a male patient are described.

The present invention relates to pharmaceutical products and methods for the treatment of infertility in male patients.

### BACKGROUND

Infertility affects approximately 15% of couples globally, with male infertility estimated to contribute to around 50% of the cases. (Agarwal, et al., Reprod Biol Endocrinol 13, 37 (2015)). However, in up to 40% of the cases of male infertility, the cause of infertility cannot be identified; many of these cases result in a diagnosis of idiopathic infertility. (Sabanegh et al., Male infertility in CAMPBELL-WALSH UROLOGY 10th ed. (Campbell et al., ed.) (Saunders Elsevier, 2012) 616-647). Idiopathic male infertility (also known as idiopathic oligoasthenoteratozoospermia) generally refers to an unexplained reduction in sperm quality.

FSH has been used for many years in the treatment of female infertility, both to promote ovulation allowing natural conception or conception after intrauterine insemination and to induce multiple follicular growth to obtain sufficient oocytes for assisted reproductive technologies (ART). Approved recombinant FSH (rFSH) products for ovarian stimulation, include follitropin alfa (GONAL-F®, Merck Serono/EMD Serono) and follitropin beta (PUREGON®/FOLLISTIM®, MSD/Schering-Plough), derived from a Chinese Hamster Ovary (CHO) cell line. The present applicants have developed a human cell line-derived rFSH (follitropin delta, also known as FE 999049), which is the subject of International Patent Application No. PCT/GB2009/000978, published as WO2009/127826A. The European Commission (EC) granted marketing authorisation for REKOVELLE® (follitropin delta) in December 2016, for use in controlled ovarian stimulation for the development of multiple follicles in women undergoing assisted reproductive technologies (ART), such as an in vitro fertilisation (IVF) cycle or intracytoplasmic sperm injection (ICSI) cycle. REKOVELLE® is the first rFSH to be derived from a human cell line (the PER.C6® cell line). The REKOVELLE® (follitropin delta) product is produced by methods disclosed in International Patent Application No. PCT/GB2009/000978.

However, there is a need for improved treatment of infertility in males, particularly males with idiopathic infertility, to improve the chance of spontaneous pregnancy in their female partners. There also is a need for improved treatment of male factor infertility, testosterone deficiency in a male patient, and idiopathic oligospermia.

### SUMMARY

The pharmaceutical products and methods described herein rely on the therapeutic value of rFSH that includes α2,3-sialylation and α2,6-sialylation in the treatment of infertile men, particularly men with idiopathic infertility, on account of the crucial role played by FSH in spermatogenesis. Thus, the use of rFSH that includes α2,3-sialylation and α2,6-sialylation to increase sperm production in male infertility patients is disclosed herein as an alternative treatment to the current standard approach, intracytoplasmic sperm injection (ICSI).

A previous meta-analysis of controlled clinical trials of FSH treatment of men with idiopathic infertility reported an improvement in pregnancy rates, both spontaneously and after ART, when men were treated with recombinant (GONAL-F®) or urinary-derived FSH (Santi et al., Endocr Connect. 2015; 4: R46-58). However, study heterogeneity, high risk of bias, and lack of precise criteria to select the population most likely to respond to treatment limited the strength of the authors' conclusions, and highlighted the need for further research in this area, including clinical trials to define who will and who will not respond to FSH treatment.

A more recent meta-analysis evaluating FSH treatment effects on sperm parameters concluded that high FSH doses (700-1050 IU/week) improved sperm concentration, total sperm count and progressive motility, whereas lower FSH doses (up to 262.5 IU/week) increased only sperm motility, and intermediate FSH doses (350-525 IU/week) improved sperm concentration with only a trend to increased sperm count and motility (Canarella et al., Asian J Androl. 2020; 22:309 16). The reported analysis did not identify different efficacy between urinary-derived FSH and recombinant FSH.

Results of a dose-range trial conducted in 354 men, concluded that men with idiopathic oligozoospermia treated with the FSH product Urofollitropin for Injection (Livzon Pharmaceutical Group Co., Ltd., Zhuhai, China) had a substantially increased sperm count when treated with at least 200 IU every other day, with those results first observed at the beginning of the third month of treatment (Ding et al., Clin Endocrinol. 2015; 83:866-71). Improvements in both sperm morphology and forward motility were reported beginning at the fifth month of treatment, and it was reported that administration of 300 IU every other day for 5 months could substantially improve the spontaneous pregnancy rate as well as the ART pregnancy rate. Although the authors refer to the FSH used as “recombinant FSH” and “rFSH,” it is believed that the identified FSH product actually used in the study (Urofollitropin for Injection) was purified, urinary-derived FSH, not a recombinant product.

Against this backdrop, the present inventors believe the treatment of men with daily doses of 11-13 μg of rFSH that includes α2,3-sialylation and α2,6-sialylation (e.g., 12 μg FE 999049) will be effective in the treatment of idiopathic infertility, to improve the chance of spontaneous pregnancy observed in their female partners in comparison to placebo. The present inventors also believe this dosing regimen will be effective in the treatment of male factor infertility, testosterone deficiency in a male patient, and idiopathic oligospermia. The treatment also may be effective to improve one or more or all of semen volume, sperm concentration, total sperm count, sperm motility, and sperm morphology, or be effective to reduce semen DNA fragmentation, of sperm/semen of the treated male patient. The treatment also may be effective to improve the chance of a positive beta-human chorionic gonadotropins (BhCG) urine test in a female partner of the treated subject in comparison to men treated with placebo (inactive treatment). The treatment also may be effective to reduce the time to spontaneous pregnancy in a female partner of the treated subject in comparison to men treated with placebo (as may be assessed, e.g., by one or more of calendar time and number of menstrual cycles).

This dosing regimen provides a higher dose of FSH than used previously (e.g., greater than the dosing regimen of 300 IU FSH every other day reported in Ding), and involves more frequent dosing (i.e., daily dosing). Thus, the dosing regimen described herein will result in increased exposure to FSH. While FSH has been shown to act in a dose-dependent manner in other contexts, e.g., in the treatment of infertility in women, the impact of such high, daily dosing in men has not previously been studied. For example, while the pituitary gland releases FSH in a pulsatile manner, the effect of daily FSH dosing resulting in relatively stable serum concentrations of FSH in men has not been reported.

In women, 150 IU/day follitropin alfa (GONAL-FR) corresponds to 10 μg/day FE 999049 (Arce et al., RBMO. 2020; 41 (4): 616-22). The present inventors therefore believe the daily dose of, e.g., 12 μg FE 999049, described herein will provide a 20% higher exposure level than the previously assessed dose of 300 IU every other day, i.e., the dosing regimen described herein will provide exposure approximately equivalent to 180 IU/day.

The present inventors also believe the daily dosing described herein will result in smaller fluctuations (i.e., more consistent exposure) in serum FSH concentrations than treatment every other day for two reasons: firstly, rFSH that includes α2,3-sialylation and α2,6-sialylation, such as the human cell line-derived FSH product FE99049, has a lower clearance rate than CHO-cell derived products; and secondly, administration every day is expected to smooth out fluctuations as compared to, e.g., administration of twice the dose every other day. As noted above, however, the pituitary gland releases FSH in a pulsatile manner, and the effect of more steady FSH dosing in men has not been reported.

On the other hand, supraphysiological FSH levels in men are believed to be safe and well-tolerated because FSH interacts only via the specific FSH receptor (FSHR) in the Sertoli cells of the testes (Simoni et al., Andrology 2020; 8 (3): 535-44). Further, to date, no evidence of FSHR downregulation in response to FSH stimulation has been reported and no direct role for FSH in bone turnover or metabolic functions in humans has been identified.

Based on the foregoing, a proof-of-concept (PoC) clinical trial is described herein below. The clinical trial will assess the treatment of men with idiopathic infertility using a daily dose of 11-13 μg FE 999049 (e.g., 12 μg FE 999049) for 6 months, with regard to improving the chance of spontaneous pregnancy observed in their female partners in comparison to placebo. While not wanting to be bound by theory, for reasons including those outlined above, the present inventors believe the dosing regimen described herein using rFSH that includes α2,3-sialylation and α2,6-sialylation will be effective in improving the chance of spontaneous pregnancy observed in their female partners in comparison to placebo. For example, the present inventors believe the results of the clinical trial will demonstrate that a daily dose of 11-13 μg FE 999049 (e.g., 12 μg FE 999049) will provide a significantly greater FSH exposure level than is provided by daily administration of conventional (e.g., CHO-cell derived) rFSH. The present inventors also believe the dosing regimen described herein of rFSH that includes α2,3-sialylation and α2,6-sialylation, e.g., FE 999049, will result in smaller fluctuations in serum FSH levels than treatment with 300 IU FSH every other day (e.g., the dosing regimen reported in Ding 2015, supra). Thus, while Ding reported efficacy of its 300 IU FSH every other day regimen, the present inventors believe the dosing regimen described herein of rFSH that includes α2,3-sialylation and α2,6-sialylation (e.g., FE 999049) may prove to be more effective. Further, the present inventors believe a daily dose of 11-13 μg of rFSH that includes α2,3-sialylation and α2,6-sialylation (e.g., 12 μg FE 999049) will be safe and well-tolerated in male patients.

Accordingly, present inventors believe the dosing regimen described herein can improve the chance of spontaneous pregnancy observed in the female partners of the treated male patients in comparison to placebo. The present inventors also believe this dosing regimen will be effective in the treatment of male factor infertility, testosterone deficiency in a male patient, and idiopathic oligospermia. The treatment also may be effective to improve one or more or all of semen volume, sperm concentration, total sperm count, sperm motility, and sperm morphology, or be effective to reduce semen DNA fragmentation, of sperm/semen of the treated male patient. The treatment also may be effective to improve the chance of a positive beta-human chorionic gonadotropins (BhCG) urine test in a female partner of the treated subject in comparison to men treated with placebo. The treatment also may be effective to reduce the time to spontaneous pregnancy in a female partner of the treated subject in comparison to men treated with placebo (as may be assessed, e.g., by one or more of calendar time and number of menstrual cycles).

Most studies of FSH treatment to improve sperm parameters have a treatment period of at least 3 months. See, e.g., Ding, supra. This is because a complete spermatogenic cycle has been estimated to take 60-70 days. See, e.g., Heller, Science, 140:184-86 (1963). And the time for transport in the ductal system has been estimated to take another 20-30 days. See, e.g., Shaw's Textbook of Gynaecology, p. 201 (Padubidri and Daftary, eds.) (15th ed., 2011). However, there is emerging evidence that these time periods may be shorter. Thus, the present inventors believe that the treatments described herein may show a treatment effect in a shorter time period, such as 30 days or one month (e.g., one medical month of 28 days). Thus, in some aspects, the treatments described herein have a treatment period of 28 days, 30 days, one month, or longer, as discussed further below.

In accordance with some aspects, there are provided compositions comprising rFSH for use in the treatment of idiopathic infertility in a male patient, wherein the rFSH includes α2,3-sialylation and α2,6-sialylation, wherein the rFSH is for administration to the patient at a daily dose of 11-13 μg (e.g., 12 μg). The infertility may be at least one sperm-related infertility selected from azoospermia, oligospermia, asthenozoospermia, teratozoospermia, parvisemia, spermatocytopenia and malformation. In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m2. In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there are provided compositions comprising rFSH for use in the treatment of infertility in a male patient, wherein the rFSH includes α2,3-sialylation and α2,6-sialylation wherein the rFSH is for administration to the patient at a daily dose of 11-13 μg (e.g., 12 μg). The infertility may be at least one sperm-related infertility selected from azoospermia, oligospermia, asthenozoospermia, teratozoospermia, parvisemia, spermatocytopenia and malformation. In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m2. In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there are provided compositions composition comprising rFSH for use in the treatment of male factor infertility in a patient, wherein the rFSH includes α2,3-sialylation and α2,6-sialylation wherein the rFSH is for administration to the patient at a daily dose of 11-13 μg (e.g., 12 μg). The infertility may be at least one sperm-related infertility selected from azoospermia, oligospermia, asthenozoospermia, teratozoospermia, parvisemia, spermatocytopenia and malformation. In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there are provided compositions comprising rFSH for use in the treatment of testosterone deficiency in a male patient, wherein the rFSH includes α2,3-sialylation and α2,6-sialylation and wherein the rFSH is for administration to the patient at a daily dose of 11-13 μg (e.g., 12 μg). In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there are provided compositions comprising rFSH for use in the treatment of idiopathic oligospermia in a male patient, wherein the rFSH includes α2,3-sialylation and α2,6-sialylation and wherein the rFSH is for administration to the patient at a daily dose of 11-13 μg (e.g., 12 μg). In some aspects, the rFSH includes α2,3-sialylation and 2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there is provided a method of treatment of infertility, including idiopathic infertility and male factor infertility, in a male patient, comprising; administering to the patient rFSH including α2,3-sialylation and α2,6-sialylation, wherein the rFSH is administered at a dose of 11-13 μg (e.g., 12 μg) per day. The infertility may be one or more sperm-related infertility conditions selected from azoospermia, oligospermia, asthenozoospermia, teratozoospermia, parvisemia, spermatocytopenia and malformation. In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². Additionally or alternatively, in some aspects, the patient has a semen volume ≥1.4 mL. Additionally or alternatively, in some aspects, the patient has a baseline total sperm count of 5 to 39 million. Additionally or alternatively, in some aspects, the patient has a sperm concentration less than 5 million spermatozoa per mL. Additionally or alternatively, in some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. Additionally or alternatively, in some aspects, the patient has total motile sperm count of 5 to 16 million. Additionally or alternatively, in some aspects, the patient has ≥15% spermatozoa with normal morphology (an oval-shaped head, an intact midpiece and an uncoiled single tail and ability to swim well and in a straight line). Additionally or alternatively, in some aspects, the patient has baseline serum hormone concentration of follicle-stimulating hormone (FSH) of 1.5 to 8.0 IU/L. Additionally or alternatively, in some aspects, the patient has baseline serum hormone concentration of luteinising hormone (LH) of 1.2 to 7.5 IU/L. Additionally or alternatively, in some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there is provided a method of treatment of testosterone deficiency in a male patient, comprising; administering to the patient rFSH including α2,3-sialylation and α2,6-sialylation, wherein the rFSH is administered at a dose of 11-13 μg (e.g., 12 μg) per day. In some aspects, the rFSH includes α2,3-sialylation and α2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In accordance with some aspects, there is provided a method of treatment of idiopathic oligospermia in a male patient, comprising administering to the patient rFSH including α2,3-sialylation and α2,6-sialylation, wherein the rFSH is administered at a dose of 11-13 μg (e.g., 12 μg) per day. In some aspects, the rFSH includes α2,3-sialylation and 2,6-sialylation wherein 5 to 20% of the total sialylation is α2,6-sialylation. In some aspects the rFSH is produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year).

In some aspects, the patient has one or more of the following characteristics prior to treatment, e.g., at the start of treatment or within about 90 days of the start of treatment. In some aspects, the patient has a Body Mass Index (BMI)≤35 kg/m². In some aspects, the patient has a semen volume ≥1.4 mL. In some aspects, the patient has a baseline total sperm count of 5 to 39 million. In some aspects, the patient has a sperm concentration less than 5 million spermatozoa per mL. In some aspects, the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL. In some aspects, the patient has total motile sperm count of 5 to 16 million. In some aspects, the patient has ≥15% spermatozoa with normal morphology (an oval-shaped head, an intact midpiece and an uncoiled single tail and ability to swim well and in a straight line). In some aspects, the patient has baseline serum hormone concentration of follicle-stimulating hormone (FSH) of 1.5 to 8.0 IU/L. In some aspects, the patient has baseline serum hormone concentration of luteinising hormone (LH) of 1.2 to 7.5 IU/L. In some aspects, the patient has baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

Thus, there is provided a composition comprising rFSH for use in the treatment of idiopathic infertility in a male patient, wherein the rFSH includes α2,3-sialylation and α2,6-sialylation, wherein the rFSH is for administration to the patient at a daily dose of 11-13 μg (e.g., 12 μg). Additionally, there is provided a composition comprising rFSH for use in the treatment of infertility in a male patient, wherein the rFSH includes α2,3-sialylation and α2,6-sialylation, wherein the rFSH is for administration to the patient at a daily dose of 11-13 μg (e.g., 12 μg). Additionally, there is provided a composition comprising rFSH for use in the treatment of male factor infertility in a patient, wherein the rFSH includes α2,3-sialylation and α2,6-sialylation, wherein the rFSH is for administration to the patient at a daily dose of 11-13 μg (e.g., 12 μg). Additionally, there is provided a composition comprising rFSH for use in the treatment of testosterone deficiency in a male patient, wherein the rFSH includes α2,3-sialylation and α2,6-sialylation, and wherein the rFSH is for administration to the patient at a daily dose of 11-13 μg (e.g., 12 μg). Additionally, there is provided a composition comprising rFSH for use in the treatment of idiopathic oligospermia in a male patient, wherein the rFSH includes α2,3-sialylation and α2,6-sialylation, and wherein the rFSH is for administration to the patient at a daily dose of 11-13 μg (e.g., 12 μg). Additionally, there is provided a method of treating infertility in a male patient, including male factor infertility and/or idiopathic infertility, comprising administering to the patient rFSH including α2,3-sialylation and α2,6-sialylation, wherein the rFSH is administered at a dose of 11-13 μg per day, optionally at a dose of 12 μg per day. Additionally, there is provided a method of treating testosterone deficiency in a male patient, comprising administering to the patient rFSH including α2,3-sialylation and α2,6-sialylation, wherein the rFSH is administered at a dose of 11-13 μg per day, optionally at a dose of 12 μg per day. Additionally, there is provided a method of treating idiopathic oligospermia in a male patient, comprising administering to the patient rFSH including α2,3-sialylation and α2,6-sialylation, wherein the rFSH is administered at a dose of 11-13 μg per day, optionally at a dose of 12 μg per day.

In accordance with any of the foregoing, the infertility may be at least one sperm-related infertility selected from azoospermia, oligospermia, asthenozoospermia, teratozoospermia, parvisemia, spermatocytopenia and malformation.

In accordance with any of the foregoing, from 5 to 20% of the total sialylation may be α2,6-sialylation and/or the rFSH may be produced or expressed in a PERC6® cell line.

In accordance with any of the foregoing, the administration to the patient at a daily dose of 11-13 μg (e.g., 12 μg) may be for a treatment period selected from (i) at least 28 days; (ii) from 28 days to 6 months; (iii) at least 30 days; (iv) from 30 days to 6 months; (v) 28 days; (vi) 30 days; (vii) 6 weeks; (viii) 8 weeks; (ix) 2 months; (x) 10 weeks; (xi) 12 weeks; (xii) 3 months; (xiii) 14 weeks; (xiv) 16 weeks; (xv) 4 months; (xvi) 18 weeks; (xvii) 20 weeks; (xviii) 5 months; (xix) 22 weeks, (xx) 24 weeks, and (xxi) 6 months.

In accordance with any of the foregoing, the patient may have one or more or all of the following characteristics, e.g., prior to treatment: a Body Mass Index (BMI)≤35 kg/m²; a semen volume ≥1.4 mL; a baseline total sperm count of 5 to 39 million; a sperm concentration less than 5 million spermatozoa per mL; a sperm concentration less than or equal to 3 million spermatozoa per mL, for example less than or equal to 2 million spermatozoa per mL, for example less than or equal to 1 million sperm per mL, for example less than or equal to 0.5 million sperm per mL; a total motile sperm count of 5 to 16 million; ≥15% spermatozoa with normal morphology (an oval-shaped head, an intact midpiece and an uncoiled single tail and ability to swim well and in a straight line); a baseline serum hormone concentration of follicle-stimulating hormone (FSH) of 1.5 to 8.0 IU/L; a baseline serum hormone concentration of luteinising hormone (LH) of 1.2 to 7.5 IU/L; a baseline serum hormone concentration of testosterone of ≥300 ng/dl (equals ≥10.4 nmol/L).

In accordance with any of the foregoing, the treatment may be effective to provide an improved chance of spontaneous pregnancy in a female partner of the treated male patient. Additionally or alternatively, in accordance with any of the foregoing, the treatment may be effective to improve one or more or all of semen volume, sperm concentration, total sperm count, sperm motility, and sperm morphology, or is effective to reduce semen DNA fragmentation, of sperm/semen of the treated male patient. Additionally or alternatively, in accordance with any of the foregoing, the treatment may be effective to improve the chance of a positive beta-human chorionic gonadotropins (BhCG) urine test in a female partner of the treated subject in comparison to men treated with placebo. Additionally or alternatively, in accordance with any of the foregoing, the treatment may be effective to reduce the time to spontaneous pregnancy in a female partner of the treated subject in comparison to men treated with placebo.

### DETAILED DESCRIPTION

The proof-of-concept (PoC) trial described below will demonstrate efficacy of the therapeutic products and methods described herein. The inventors believe men with idiopathic infertility, after being treated with a daily dose of 11-13 μg FE 999049 (e.g., 12 μg FE 999049), will have an improved chance of spontaneous pregnancy observed in their female partners in comparison to men treated with placebo. The treatment also may be effective to improve one or more or all of semen volume, sperm concentration, total sperm count, sperm motility, and sperm morphology, or be effective to reduce semen DNA fragmentation, of sperm/semen of the treated male patient. The treatment also may be effective to improve the chance of a positive beta-human chorionic gonadotropins (BhCG) urine test in a female partner of the treated subject in comparison to men treated with placebo. The treatment also may be effective to reduce the time to spontaneous pregnancy in a female partner of the treated subject in comparison to men treated with placebo (as may be assessed, e.g., by one or more of calendar time and number of menstrual cycles).

As noted above, a treatment effect may be observed before the end of the 6 month study treatment period. Additionally, the treatment benefit to patients may persist for some period of time after treatment is discontinued, so the trial will follow outcomes after the end of the 6 month study treatment period, such as for 3 months after the end of treatment.

Therefore, provided herein are treatments and dosage regimes constructed for use to treat a male patient with infertility, including idiopathic infertility and male factor infertility, and also for treating male testosterone deficiency and idiopathic oligospermia.

### Definitions

Technical and scientific terms used herein have the meanings commonly understood by one of ordinary skill in the art of assisted reproductive technology to which the present invention pertains, unless otherwise defined. Reference is made herein to various methodologies known to those of ordinary skill in the art. Any suitable materials and/or methods known to those of ordinary skill in the art can be utilized in carrying out the present invention based on the guidance provided herein. However, specific materials and methods are described. Materials, reagents and the like to which reference is made in the following description and examples are obtainable from commercial sources, unless otherwise noted.

It is to be understood, that any definitions and terms herein defined are meant to have the same meaning and purpose in any of the aspects and embodiments of the invention unless explicitly otherwise stated not to.

As used herein the singular forms “a,” “an,” and “the” designate both the singular and the plural, unless expressly stated to designate the singular only.

As used herein the term “about” means that the number or range so modified is not limited to the exact number or range set forth, but encompass ranges around the stated number or range as will be understood by persons of ordinary skill in the art depending on the context in which the number or range is used. Unless otherwise apparent from the context or convention in the art, “about” means up to plus or minus 10% of the particular term.

As used herein “spontaneous pregnancy” is defined as vital pregnancy (documentation of at least one intrauterine gestational sac with fetal heartbeat by ultrasound).

As used herein “ongoing pregnancy” refers to pregnancy with a viable foetus and detectable foetal heartbeat at 10-11 weeks gestation, e.g., at 8-9 weeks post blastocyst/embryo transfer.

As used herein “clinical pregnancy” refers to gestation and a detectable foetal heartbeat at 5-6 weeks gestation, e.g., at 3-4 weeks post blastocyst/embryo transfer.

In the treatments, methods and uses described herein, the administration of rFSH starts on “day one” of treatment and may continue for 6 months, or longer; for example, treatment may continue for 1, 2, 3, 4, 5 or 6 months as discussed in more detail below.

As used herein the term “spermatogenesis” refers to the production or development of mature spermatozoa.

As used herein the term “azoospermia” refers to a condition where there is no measurable sperm in the ejaculate. As used herein the term “oligospermia” refers to a condition where there is a low sperm concentration in the ejaculate, for example less than 15 million sperm per mL of semen. As used herein the term “asthenozoospermia” refers to a condition wherein the sperm has poor motility, for example less than 40% motility. As used herein the term “teratozoospermia” refers to a condition wherein the sperm has a high amount of abnormal morphology wherein normal morphology is defined as an oval-shaped head, an intact midpiece and an uncoiled single tail and ability to swim well and in a straight line. As used herein the term “parvisemia” refers to a condition wherein a male has a low volume of ejaculate, for example less than 2 mL or less than 1.5 mL. As used herein the term “spermatocytopenia” refers to a reduction in the number of spermatozoa from normal levels.

Herein the terms “patient” and “subject” and “male” and “man” are used interchangeably.

In some aspects, the patient has a BMI as follows prior to treatment, e.g., at the start of treatment or within about 90 days of the start of treatment. A subject may have a BMI >15 and BMI <40 kg/m2, for example a BMI >17.5 and BMI <38 kg/m2, for example a BMI >18 and BMI <25 kg/m2, for example a BMI >20 and BMI <25 kg/m2. Thus a product or method as described herein may be used for the treatment of infertility in a patient having BMI >15 and BMI <40 kg/m2, for example a subject having BMI >17.5 and BMI <38 kg/m2, for example a subject having BMI >18 and BMI <25 kg/m2, for example a subject having BMI >20 and BMI <25 kg/m2. Thus a product or method as described herein may be used for the treatment of infertility in a patient having BMI >17.5 and BMI <32 kg/m2. BMI may be measured by methods well known in the art to identify the patient for treatment.

In some aspects, the patient has one or more of the following characteristics prior to treatment, e.g., at the start of treatment or within about 90 days of the start of treatment. The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dl (equals ≥10.4 nmol/L).

These selection criteria are designed to identify patients most likely to benefit from the treatments described herein. For example, the FSH, LH and testosterone parameters may encompass patients who have gonadotropin levels that are on the lower side of normal. The FSH, LH and testosterone thresholds may exclude patients with hypogonadotropic hypogonadism, who would not be well-served by FSH treatment alone. On the other hand, upper limits on the FSH and LH parameters may exclude patients who have high-normal to excessively high levels of gonadotropins, whose idiopathic infertility may have a non-gonadotropin etiology and for whom therapeutic efficacy of FSH (if any) could be difficult to detect.

Thus, at the time of treatment, including at the start of treatment (including within about 90 days of the start of treatment), the patient may have one or more or all of the following characteristics: a Body Mass Index (BMI)≤35 kg/m²; a semen volume ≥1.4 mL; a baseline total sperm count of 5 to 39 million; a sperm concentration less than 5 million spermatozoa per mL, for example less than 4 million spermatozoa per mL; a sperm concentration less than or equal to 3 million spermatozoa per mL, for example less than or equal to 2 million spermatozoa per mL, for example less than or equal to 1 million sperm per mL, for example less than or equal to 0.5 million sperm per mL; a total motile sperm count of 5 to 16 million, for example 5 to 10 million, for example 6 to 8 million; ≥15% spermatozoa with normal morphology (an oval-shaped head, an intact midpiece and an uncoiled single tail and ability to swim well and in a straight line (e.g., forward progression/progressive motility)); a baseline serum hormone concentration of follicle-stimulating hormone (FSH) of 1.5 to 8.0 IU/L; a baseline serum hormone concentration of luteinising hormone (LH) of 1.2 to 7.5 IU/L; a baseline serum hormone concentration of testosterone of ≥300 ng/dl (equals ≥10.4 nmol/L).

The foregoing parameters can be assessed by methodologies known in the field, such as those in accordance with World Health Organisation (WHO) standards. See, e.g., Boitrelle et al., Life (2021) 11 (12): 1368. Assessment of semen and sperm parameters may be conducted via a laboratory-conducted manual semen assessment, optionally including Computer-Assisted Sperm Analysis. Additionally or alternatively, assessment of sperm parameters may include use of a sperm testing device, such as the ExSeed® device (ExSeed Health, Denmark), or other sperm testing device, such as those reviewed in Onofre, et al., Facts Views Vis ObGyn (2021) 13 (1): 79-93). Parameters pertaining to semen and sperm characteristics may be assessed and confirmed in one sample or in multiple samples taken at the same time or different times, such as at the time of initial screening for treatment and subsequently, closer in time to the start of treatment, such as at the time of initial screening for treatment and in two consecutive samples taken within about 90 days of the start of treatment (e.g., ≥2 weeks before the start of treatment but within about 90 days of the start of treatment).

In some aspects, the patient has not undergone FSH treatment not leading to conception in their partner. Additionally or alternatively, in some aspects the patient has not undergone administration of hormonal preparations within 3 months prior to screening.

In accordance with all aspects described herein, the recombinant human follicle-stimulating hormone (rFSH) including α2,3- and α2,6-sialylation may be human cell line-derived recombinant FSH as described in more detail below. In all aspects, the recombinant FSH including α2,3- and α2,6-sialylation may be that sold under the trademark REKOVELLE® (follitropin delta) (Ferring B.V.). In all aspects, the recombinant FSH may be administered by injection, e.g., subcutaneous injection.

### Treatments

In the proof-of-concept (PoC) trial described below, the inventors will show that men with idiopathic infertility, after being treated with a daily dose of 11-13 μg FE 999049 (e.g., 12 μg FE 999049), will have an improved chance of spontaneous pregnancy in their female partners in comparison to men treated with placebo.

The treatment also may be effective to improve one or more or all of semen volume, sperm concentration, total sperm count, sperm motility, and sperm morphology, or be effective to reduce semen DNA fragmentation, of sperm/semen of the treated male patient. For example, the treatment may be effective to increase total sperm count or total motile sperm count to 50% or more over baseline.

The treatment also may be effective to improve the chance of a positive beta-human chorionic gonadotropins (BhCG) urine test in a female partner of the treated subject in comparison to men treated with placebo. The treatment also may be effective to reduce the time to spontaneous pregnancy in a female partner of the treated subject in comparison to men treated with placebo (as may be assessed, e.g., by one or more of calendar time and number of menstrual cycles).

As noted above, a treatment effect may be observed after from 28 days or 30 days to 6 months of treatment. Additionally, the treatment benefit to patients may persist for some period of time after treatment is discontinued, e.g., three months or longer after the end of treatment.

The present inventors believe the results of the trial will demonstrate that a daily dose of 11-13 μg FE 999049 (e.g., 12 μg FE 999049) will provide a significantly greater FSH exposure level (as assessed by AUC and Cₘₐₓ, e.g., being 1.7- and 1.6-fold higher, respectively) than would be achieved with daily administration of conventional (CHO-cell derived) rFSH, because of the lower clearance rate of FE 999049 (0.58 L/h for FE 999049 versus 0.99 L/h for follitropin alfa). (See, e.g., Olssen et al., J. Clin. Pharmacol. (2014) 54 (11) 1299-1307). The present inventors also believe the daily dose of FE 999049 will result in smaller fluctuations in serum FSH levels (i.e., more consistent exposure) than treatment with 300 IU FSH every other day (as reported in Ding 2015, supra). Further, the present inventors believe a daily dose of 11-13 μg FE 999049 (e.g., 12 μg FE 999049) will be safe and well-tolerated in male patients.

Accordingly, the present inventors believe the dosing regimen described herein will improve the chance of spontaneous pregnancy observed in the female partners of the treated male patients in comparison to patients treated with placebo (inactive treatment). The treatment also may be effective to improve one or more or all of semen volume, sperm concentration, total sperm count, sperm motility, and sperm morphology, or be effective to reduce semen DNA fragmentation, of sperm/semen of the treated male patient. The treatment also may be effective to improve the chance of a positive beta-human chorionic gonadotropins (BhCG) urine test in a female partner of the treated subject in comparison to men treated with placebo. The treatment also may be effective to reduce the time to spontaneous pregnancy in a female partner of the treated subject in comparison to men treated with placebo (as may be assessed, e.g., by one or more of calendar time and number of menstrual cycles).

### Therapeutic Compositions

In a first aspect, there is provided a composition (e.g., a pharmaceutical composition) comprising rFSH for use in the treatment of idiopathic infertility in a male patient, wherein the rFSH includes α2,3-sialylation and α2,6-sialylation (e.g., follitropin delta) wherein the rFSH is for administration to the patient at a daily dose of 11-13 μg (e.g., 12 μg).

The infertility may be at least one sperm-related infertility selected from azoospermia, oligospermia, asthenozoospermia, teratozoospermia, parvisemia, spermatocytopenia and malformation.

The rFSH may include α2,3-sialylation and α2,6-sialylation wherein 5 to 20% of the total sialylation is α2,6-sialylation. The rFSH may include α2,3-sialylation and α2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase. The PER.C6® cell line has been deposited at the European Collection of Authenticated Cell Cultures under ECACC deposit No. 96022940, as of Feb. 29, 1996.

As noted above, in some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In a second aspect, there is provided a composition (e.g., a pharmaceutical composition) comprising rFSH for use in the treatment of infertility in a male patient, wherein the rFSH includes α2,3-sialylation and α2,6-sialylation (e.g., follitropin delta) wherein the rFSH is for administration to the patient at a daily dose of 11-13 μg (e.g., 12 μg).

The infertility may be at least one sperm-related infertility selected from azoospermia, oligospermia, asthenozoospermia, teratozoospermia, parvisemia, spermatocytopenia and malformation.

The rFSH may include α2,3-sialylation and α2,6-sialylation wherein 5 to 20% of the total sialylation is α2,6-sialylation. The rFSH may include α2,3-sialylation and α2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dL (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In a further aspect, there is provided a composition (e.g., a pharmaceutical composition) comprising rFSH for use in the treatment of male factor infertility in a patient, wherein the rFSH includes α2,3-sialylation and α2,6-sialylation (e.g., follitropin delta) wherein the rFSH is for administration to the patient at a daily dose of 11-13 μg (e.g., 12 μg).

The infertility may be at least one sperm-related infertility selected from azoospermia, oligospermia, asthenozoospermia, teratozoospermia, parvisemia, spermatocytopenia and malformation.

The rFSH may include α2,3-sialylation and α2,6-sialylation wherein 5 to 20% of the total sialylation is α2,6-sialylation. The rFSH may include α2,3-sialylation and α2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase. The PER.C6® cell line has been deposited at the European Collection of Authenticated Cell Cultures under ECACC deposit No. 96022940, as of Feb. 29, 1996.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dl (equals ≥10.4 nmol/L). As used herein, a “baseline” value refers to a value prior to treatment as described herein. In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In a further aspect, there is provided a composition (e.g., a pharmaceutical composition) comprising rFSH for use in the treatment of testosterone deficiency in a male patient, wherein the rFSH includes α2,3-sialylation and α2,6-sialylation (e.g., follitropin delta) wherein the rFSH is for administration to the patient at a daily dose of 11-13 μg (e.g., 12 μg).

The rFSH may include α2,3-sialylation and α2,6-sialylation wherein 5 to 20% of the total sialylation is α2,6-sialylation. The rFSH may include α2,3-sialylation and α2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase. The PER.C6® cell line has been deposited at the European Collection of Authenticated Cell Cultures under ECACC deposit No. 96022940, as of Feb. 29, 1996.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

In a further aspect, there is provided a composition (e.g., a pharmaceutical composition) comprising rFSH for use in the treatment of idiopathic oligospermia in a male patient, wherein the rFSH includes α2,3-sialylation and α2,6-sialylation (e.g., follitropin delta) wherein the rFSH is for administration to the patient at a daily dose of 11-13 μg (e.g., 12 μg).

The rFSH may include α2,3-sialylation and α2,6-sialylation wherein 5 to 20% of the total sialylation is α2,6-sialylation. The rFSH may include α2,3-sialylation and 2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase. The PER.C6® cell line has been deposited at the European Collection of Authenticated Cell Cultures under ECACC deposit No. 96022940, as of Feb. 29, 1996.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dl (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

### Methods of Treatment

In a further aspect, there are provided methods of treatment of infertility in a male patient, comprising administering to the patient rFSH including α2,3-sialylation and α2,6-sialylation, wherein the rFSH is administered at a dose of 11-13 μg (e.g., 12 μg) per day. As noted above, the rFSH may be administered by injection, e.g., subcutaneous injection.

The treatment of infertility may be treatment of idiopathic infertility. The treatment of infertility may be treatment of male factor infertility. The infertility may be one or more sperm-related infertility conditions selected from azoospermia, oligospermia, asthenozoospermia, teratozoospermia, parvisemia, spermatocytopenia and malformation.

In a further aspect, there are provided methods for treating male testosterone deficiency, comprising administering to a male patient rFSH including α2,3-sialylation and α2,6-sialylation, wherein the rFSH is administered at a dose of 11-13 μg (e.g., 12 μg) per day. As noted above, the rFSH may be administered by injection, e.g., subcutaneous injection.

In a further aspect, there are provided methods for treating idiopathic oligospermia in a male patient, comprising administering to the patient rFSH including α2,3-sialylation and α2,6-sialylation, wherein the rFSH is administered at a dose of 11-13 μg (e.g., 12 μg) per day. As noted above, the rFSH may be administered by injection, e.g., subcutaneous injection.

In any of these methods of treatment, the rFSH may include α2,3-sialylation and α2,6-sialylation wherein 5 to 20% of the total sialylation is α2,6-sialylation. The rFSH may include α2,3-sialylation and α2,6-sialylation wherein 80 to 90% of the total sialylation is α2,3-sialylation and wherein 10 to 20% of the total sialylation is α2,6-sialylation (e.g., follitropin delta, available from Ferring B.V.). The rFSH may be rFSH produced or expressed in a human cell line, for example a PERC6® cell line, for example a PERC6® cell line which has been modified with an α2,3-sialyltransferase. The PER.C6® cell line has been deposited at the European Collection of Authenticated Cell Cultures under ECACC deposit No. 96022940, as of Feb. 29, 1996.

In some aspects, the administration to the patient at a daily dose of 11-13 μg (e.g., 12 μg) is for a treatment period of at least 28 days (e.g., one medical month). In some aspects, the administration to the patient at a daily dose of 11-13 μg (e.g., 12 μg) is for a treatment period of at least 30 days. In some aspects, the administration to the patient at a daily dose of 11-13 μg (e.g., 12 μg) is for a treatment period of from 28 days to 6 months. In some aspects, the administration to the patient at a daily dose of 11-13 μg (e.g., 12 μg) is for a treatment period of from 30 days to 6 months. In some aspects, the administration to the patient at a daily dose of 11-13 μg (e.g., 12 μg) is for a treatment period selected from 28 days, 30 days, 6 weeks, 8 weeks, 2 months, 10 weeks, 12 weeks, 3 months, 14 weeks, 16 weeks, 4 months, 18 weeks, 20 weeks, 5 months, 22 weeks, 24 weeks, and 6 months, or longer. In some aspects, the administration to the patient at a daily dose of 11-13 μg (e.g., 12 μg) is for 28 days. In some aspects, the administration to the patient at a daily dose of 11-13 μg (e.g., 12 μg) is for 30 days. In some aspects, the administration to the patient at a daily dose of 11-13 μg (e.g., 12 μg) is for 2 months. In some aspects, the administration to the patient at a daily dose of 11-13 μg (e.g., 12 μg) is for 3 months. In some aspects, the administration to the patient at a daily dose of 11-13 μg (e.g., 12 μg) is for 4 months. In some aspects, the administration to the patient at a daily dose of 11-13 μg (e.g., 12 μg) is for 5 months. In some aspects, the administration to the patient at a daily dose of 11-13 μg (e.g., 12 μg) is for 6 months. In some aspects, the administration to the patient at a daily dose of 11-13 μg (e.g., 12 μg) is for a treatment period of at least six months (including for a treatment period of at least one year). It will be appreciated that the administration of rFSH to the patient at a daily dose of 11-13 μg (e.g., 12 μg) may be required for a longer period of time, up to 18 months, for example to achieve spermatogenesis. On the other hand, the treatment benefit to patients may persist for some period of time after treatment is discontinued, so shorter treatment periods may be efficacious.

In some aspects, the patient has one or more of the following characteristics prior to treatment, e.g., at the start of treatment or within about 90 days of the start of treatment. The patient may have a Body Mass Index (BMI)≤35 kg/m². The patient may have a semen volume ≥1.4 mL. The patient may have a baseline total sperm count of 5 to 39 million. The patient may have a sperm concentration less than 5 million spermatozoa per mL, for example less than 4 million spermatozoa per mL. The patient may have a sperm concentration less than or equal to 3 million spermatozoa per mL, for example less than or equal to 2 million spermatozoa per mL, for example less than or equal to 1 million sperm per mL, for example less than or equal to 0.5 million sperm per mL. The patient may have a total motile sperm count of 5 to 16 million, for example 5 to 10 million, for example 6 to 8 million. The patient may have ≥15% spermatozoa with normal morphology (an oval-shaped head, an intact midpiece and an uncoiled single tail and ability to swim well and in a straight line). The patient may have a baseline serum hormone concentration of follicle-stimulating hormone (FSH) of 1.5 to 8.0 IU/L. The patient may have a baseline serum hormone concentration of luteinising hormone (LH) of 1.2 to 7.5 IU/L. The patient may have a baseline serum hormone concentration of testosterone of ≥300 ng/dL (equals ≥10.4 nmol/L). In some aspects, the patient has not undergone FSH treatment not leading to conception in their partner. Additionally or alternatively, in some aspects the patient has not undergone administration of hormonal preparations within 3 months prior to screening.

In accordance with some aspects, the treatment is effective to improve the chance of spontaneous pregnancy observed in the female partners of the treated male patients in comparison to placebo. In accordance with some aspects, the treatment is effective to improve one or more or all of semen volume, sperm concentration, total sperm count, sperm motility, and sperm morphology, or be effective to reduce semen DNA fragmentation, of sperm/semen of the treated male patient. In accordance with some aspects, the treatment is effective to improve the chance of a positive beta-human chorionic gonadotropins (BhCG) urine test in a female partner of the treated subject in comparison to men treated with placebo. In accordance with some aspects, the treatment is effective to reduce the time to spontaneous pregnancy in a female partner of the treated subject in comparison to men treated with placebo (as may be assessed, e.g., by one or more of calendar time and number of menstrual cycles).

Recombinant FSH and rFSH Compositions

The methods and compositions described herein use recombinant FSH (rFSH) that includes α2,3-sialylation and α2,6-sialylation. FSH comprises a 92 amino acid alpha subunit, also common to the other glycoprotein hormones LH and chorionic gonadotropin (CG), and a 111 amino acid beta subunit unique to FSH that confers the biological specificity of the hormone. Each subunit is post translationally modified by the addition of complex carbohydrate residues. Both subunits carry two sites for N-linked glycan attachment, the alpha sub-unit at amino acids 52 and 78 and the beta sub-unit at amino acid residues 7 and 24. FSH is thus glycosylated to about 30% by mass.

The glycosylation of a given rFSH product reflects the range of glycosyl-transferases present in the host cell line. Commercially available rFSH products derived from engineered CHO cells have a more limited range of glycan modifications than those found on the natural human products. Examples of the reduced glycan heterogeneity found in CHO cell-derived rFSH include a lack of bisecting glucosamine and a reduced content of core fucosylation and acetyl lactosamine extensions. In addition, CHO cells are only able to add sialic acid using the α2,3-linkage; CHO cell-derived rFSH only includes α2,3-linked sialic acid and does not include α2,6-linked sialic acid. Thus, CHO cell-derived rFSH is different from naturally produced human FSH (e.g., human pituitary/serum/urinary FSH) which contains glycans with a mixture of α2,3- and α2,6-linked sialic acid, with a predominance of the former.

As noted above, the present applicants have developed a human cell line-derived rFSH which is the subject of International Patent Application No. PCT/GB2009/000978, published as WO2009/127826A, and also approved by the EC as REVOKELLE® (follitropin delta, also known as FE 999049). Recombinant FSH with a mixture of both α2,3- and α2,6-linked sialic acid was made by engineering a human cell line to express both rFSH and α2,3-sialyltransferase. The amino acid sequence of the human cell line-derived recombinant FSH which is the subject of International Patent Application No. PCT/GB2009/000978, published as WO2009/127826A (e.g., FE 999049/follitropin delta), is the same as the native human FSH sequence, but FE 999049 has a different glycosylation pattern. The recombinantly expressed product is highly acidic and carries a mix of both α2,3- and α2,6-linked sialic acids; the latter provided by the endogenous sialyl transferase activity. It was found that the type of sialic acid linkage, α2,3- or α2,6-, can have a dramatic influence on biological clearance of FSH. Thus REVOKELLE® (e.g., FE 999049) may be more biologically appropriate (and more biologically active) compared to CHO cell-derived recombinant products that have only α2,3-linked sialic acid and have decreased sialic acid content.

Thus, the recombinant FSH that includes α2,3-sialylation and α2,6-sialylation used in accordance with the methods and compositions described herein may be produced or expressed in a human cell line, such as a PER.C6® cell line. As noted above, the PER.C6R cell line has been deposited at the European Collection of Authenticated Cell Cultures under ECACC deposit No. 96022940, as of Feb. 29, 1996. The recombinant FSH may be produced or expressed in a PER.C6® cell line, a PER.C6® derived cell line or a modified PER.C6® cell line. Recombinant FSH which is produced or expressed in a PER.C6® cell line will include some α2,6-linked sialic acids (α2,6 sialylation) provided by endogenous 2,6-sialyl transferase activity (of the cell line) and will include some α2,3-linked sialic acids (α2,3 sialylation) provided by endogenous 2,3-sialyl transferase activity. The cell line may be modified using α2,3-sialyltransferase (e.g. to increase the amount of α2,3 sialylation on the product rFSH). The cell line may be modified using α2,6-sialyltransferase (e.g. to increase the amount of α2,6 sialylation on the product rFSH). Alternatively or additionally, the recombinant FSH may include α2,6-linked sialic acids (α2,6 sialylation) provided by endogenous sialyl transferase activity (of the cell line).

The recombinant FSH used in the methods and compositions described herein may include α2,3- and α2,6-sialylation. The recombinant FSH for use according to the invention may have 1% to 99% of the total sialylation being α2,3-sialylation. The recombinant FSH for use according to the invention may have 1% to 99% of the total sialylation being α2,6-sialylation. The recombinant FSH may have 1% to 50% of the total sialylation as α2,6-sialyation, and 50% to 99% of the total sialylation as 2,3-sialyation. For example, 80% to 95%, for example 80% to 90%, for example 82% to 89%, for example 85% to 89% of the total sialylation may be α2,3-sialylation. For example, 5% to 20%, for example 10% to 20%, for example 11% to 18%, for example 11% to 15%, of the total sialylation may be α2,6-sialylation. In an example, the recombinant FSH has 5% to 20% of the total sialylation as α2,6-sialyation, and 80% to 95% of the total sialylation as 2,3-sialyation. In another example, the recombinant FSH has 50% to 80% of the total sialylation as α2,6-sialyation, and 20% to 50% of the total sialylation as 2,3-sialyation.

Herein, by “sialylation”, it is meant the amount of sialic residues present on the recombinant FSH carbohydrate structures. Consistent with usage in the art, α2,3-sialylation means sialylation at the 2,3 position and α2,6 sialylation means sialylation at the 2,6 position. Thus “% of the total sialylation may be a 2,3 sialylation” refers to the % of the total number of sialic acid residues present in the FSH which are sialylated in the 2,3 position. The term “% of the total sialylation being α2,6-sialylation” refers to the % of the total number of sialic acid residues present in the FSH which are sialylated in the 2,6 position.

In all aspects, the rFSH may be present as a single isoform or as a mixture of isoforms.

The composition may be a pharmaceutical composition. The pharmaceutical composition is for the treatment of infertility.

The recombinant FSH, composition, or pharmaceutical composition can be formulated into well-known compositions for any route of drug administration, and typically is formulated for and administered by subcutaneous injection. A typical composition comprises a pharmaceutically acceptable carrier, such as aqueous solution, nontoxic excipients, including salts and preservatives, buffers and the like, as described in Remington's Pharmaceutical Sciences fifteenth edition (Matt Publishing Company, 1975), at pages 1405 to 1412 and 1461-87, and the national formulary XIV fourteenth edition (American Pharmaceutical Association, 1975), among others. For example, the recombinant FSH, composition or pharmaceutical composition can be formulated for injection, such as for subcutaneous injection.

Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate.

The compositions of the present invention may also comprise additives such as but not limited to preservatives, wetting agents, emulsifying agents, surfactants and dispersing agents. Antibacterial and antifungal agents can be included to prevent growth of microbes and includes, for example, m-cresol, benzyl alcohol, paraben, chlorobutanol, phenol, sorbic acid, and the like. If a preservative is included, benzyl alcohol, phenol and/or m-cresol are preferred; however, the preservative is by no means limited to these examples. Furthermore, it may be desirable to include isotonic agents such as sugars, sodium chloride, amino acids and the like.

The pH and exact concentration of the various components of the pharmaceutical composition are adjusted in accordance with routine practice in this field. See GOODMAN and GILMAN's THE PHARMACOLOGICAL BASIS FOR THERAPEUTICS, 7th ed. In a typical embodiment, the recombinant FSH, composition or medicament are supplied as compositions for parenteral administration, e.g., by subcutaneous injection. General methods for the preparation of parenteral formulations, including formulations for administration by subcutaneous injection, are known in the art and are described in REMINGTON; THE SCIENCE AND PRACTICE OF PHARMACY, supra, at pages 780-820. The compositions can be supplied in liquid formulation or as a solid which will be mixed with a sterile injectable medium just prior to administration. The compositions may be supplied in dosage unit form for ease of administration and uniformity of dosage.

For example, the composition or medicament may comprise recombinant FSH and one or more of polysorbate 20, L-methionine, phenol, and arginine hydrochloride. Such a composition may be formulated for injection, such as for subcutaneous injection. For example, the composition or medicament may be the REKOVELLE® formulation (rFSH that include α2,3- and α2,6-sialylation with excipients phenol, polysorbate 20, L-methionine, sodium sulphate decahydrate, disodium phosphate dodecahydrate, phosphoric acid [concentrated, for pH-adjustment], sodium hydroxide [for pH-adjustment], and water for injection).

Injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use. Injectable formulations can be supplied in any suitable container, e.g., vial, pre-filled syringe, injection cartridges, and the like.

The recombinant FSH, composition, or medicament may be formulated for single use or for multiple uses (multiple dose). If the recombinant FSH, composition, or medicament is formulated for multiple use, typically one or more preservatives is included. If a preservative is included, benzyl alcohol, phenol or m-cresol, are preferred; however, the preservative is by no means limited to these examples. The single use or multiple use formulated composition or medicament may further comprise an amino acid or combination of amino acids. Typically, the amino acid is arginine, for example, added as arginine or more typically arginine hydrochloride.

The recombinant FSH, composition, or medicament may be included in a container such as a vial, prefilled cartridge (e.g., for single administration or multiple use) or an injection device such as a “pen” for e.g., administration of multiple doses. The recombinant FSH, composition, or medicament may be provided in a container (e.g. an administration “pen”) designed to deliver one or more doses described herein, e.g., one or more pre-determined doses of 11-13 μg (e.g., 12 μg) rFSH. For example, a prefilled cartridge or pen may contain one or more pre-determined doses of 11-13 μg (e.g., 12 μg) rFSH.

The recombinant FSH, composition or pharmaceutical composition may be a formulation (e.g., injectable formulation) including rFSH.

The recombinant FSH, composition or medicament can be supplied in any appropriate package. For example, a composition or medicament can include a number of containers (e.g., pre-filled syringes or vials) containing rFSH. The syringes or vials may be packaged in a blister package or other means to maintain sterility. Any composition or medicament can optionally include instructions for using the FSH formulation.

In a further aspect there is provided the use of recombinant follicle-stimulating hormone (FSH) that includes α2,3- and α2,6-sialylation in the manufacture of a medicament for the uses as described herein.

Further aspects are illustrated in the following examples, which are not limiting in any respect.

### EXAMPLES

The following examples use REKOVELLE® (follitropin delta), which is a recombinant FSH that includes α2,3- and α2,6-sialylation expressed in a PER.C6® cell line engineered by the methods disclosed in WO2013/020996 and WO2009/127826A.

The Marketing Authorisation holder for REKOVELLE® is Ferring Pharmaceuticals A/S of Kay Fiskers Plads 11, 2300 Copenhagen S, Denmark, and REKOVELLE® is available in the UK from Ferring Pharmaceuticals of Drayton Hall, Church Road, West Drayton, UB7 7PS, UK.

The active substance in REKOVELLE® is follitropin delta (FE999049). REKOVELLE® is highly sialylated and includes α2,3- and α2,6-sialylation, with about 85% to 90% of the total sialylation being α2,3-sialylation and about 10% to 15% of the total sialylation being α2,6-sialylation.

REKOVELLE® is a clear and colourless solution for injection (injection). One mL of solution contains 33.3 micrograms of follitropin delta in each mL of solution. The other ingredients are phenol, polysorbate 20, L-methionine, sodium sulphate decahydrate, disodium phosphate dodecahydrate, concentrated phosphoric acid, sodium hydroxide and water for injections.

### Example 1: A Trial to Compare Efficacy and Safety of Follitropin Delta Versus Placebo (Inactive Treatment) in the Treatment of Men with Idiopathic Infertility (Unexplained Reduction of Semen Quality) (ADAM)

### Introduction

The ADAM trial will investigate whether men with idiopathic infertility, after being treated with a daily dose of 12 μg recombinant follicle-stimulating hormone (rFSH) for 6 months, have improved chance of spontaneous pregnancy in their female partners in comparison to men treated with placebo (inactive treatment). The treatment benefit to patients may persist for some period of time after treatment is discontinued, so the trial will follow outcomes after the end of the 6 month study treatment, i.e., for 3 months after the end of treatment. As noted above, a treatment effect may be observed in a shorter time period, such as 30 days or one month. The treatment also may be effective to improve one or more or all of semen volume, sperm concentration, total sperm count, sperm motility, and sperm morphology, or be effective to reduce semen DNA fragmentation, of sperm/semen of the treated male patient. The treatment also may be effective to improve the chance of a positive beta-human chorionic gonadotropins (BhCG) urine test in a female partner of the treated subject in comparison to men treated with placebo (inactive treatment). The treatment also may be effective to reduce the time to spontaneous pregnancy in a female partner of the treated subject in comparison to men treated with placebo (as may be assessed by one or more of calendar time and number of menstrual cycles). The treatment also may be effective to improve the chance of a positive beta-human chorionic gonadotropins (BhCG) urine test in a female partner of the treated subject in comparison to men treated with placebo. The treatment also may be effective to reduce the time to spontaneous pregnancy in a female partner of the treated subject in comparison to men treated with placebo (as may be assessed, e.g., by one or more of calendar time and number of menstrual cycles).

### Methods

This is a randomised, double-blind, placebo-controlled, multiregional trial assessing the efficacy and safety of FE 999049 in men with idiopathic infertility. Patients will be enrolled in approximately 25 sites in approximately 7 countries across Europe and North America. All participants will provide written, informed consent prior to undergoing any study related procedures.

### Study Participants

It is planned to enroll 400 men 18-50 years of age inclusively (at the time of consent form signature) with a history of infertility for 12-60 months with their current partner. Their current partner must be a pre-menopausal woman aged 18-35 years with no contraindications to pregnancy, a regular menstrual cycles of 21-35 days, and no history or current condition of pelvic inflammatory disease, endometriosis stage II-IV by definite or empirical diagnosis, or tubal ligation.

Additional inclusion criteria for the male subjects are body mass index (BMI)≤35 kg/m², total sperm count 5-39 million at screening (and confirmed by two consecutive samples taken ≥2 weeks before randomisation), total motile sperm count of 5-16 million at screening (and confirmed by two consecutive samples taken ≥2 weeks before randomisation), semen volume ≥1.4 mL at screening (and confirmed by two consecutive samples taken ≥2 weeks before randomisation), serum follicle-stimulating hormone (FSH) levels of 1.5-8.0 IU/L at screening, serum luteinising hormone (LH) levels of 1.2-7.5 IU/L at screening, serum total testosterone levels of ≥300 ng/dl (equals ≥10.4 nmol/L) at screening. Exclusion criteria include previous FSH treatment not leading to conception and administration of hormonal preparations within 3 months prior to screening. The first man was enrolled in August 2022 and 400 will be randomized at sites in the United States and European countries.

### Study Procedures

Subjects will be screened within 90 days prior to randomisation for compliance with the inclusion and exclusion criteria. Approximately 200 participants will be randomised to receive once daily subcutaneous administration of 12 μg FE 999049 and approximately 200 participants to receive placebo treatment (Table 1).

TABLE 1
The Investigational Medicinal Products (IMPs) of the ADAM trial

| | | Active ingredient, | |
|---|---|---|---|
| | Drug | route of administration, | |
| IMP name | type | and concentration | Dose |
| FE 999049 | rFSH | Pre-filled pen containing | Daily dose |
| (follitropin delta) | | 72 μg FE 999049 in | of 12 μg |
| | | 2.16 mL solution, | FE 999049 |
| | | subcutaneous injection | for 6 months |
| Placebo | Placebo | Pre-filled pen containing | Daily dose |
| | | 2.16 mL solution, | for 6 months |
| | | subcutaneous injection | |

All randomised subjects will continue treatment for 6 months with follow up for an additional 3 months. 6 months has been selected as the treatment duration to ensure subject exposure to FE 999049 during at least two complete spermatogenic cycles, to allow for a thorough assessment of the therapeutic potential of FE 999049. Subjects will remain in the trial for 9 months regardless of whether or not spontaneous pregnancy is established at earlier time points. Should the couple experience a pregnancy loss, subjects will also remain in the trial.

In addition to partner pregnancy outcomes, semen parameters (e.g., semen volume, sperm concentration, total sperm count, sperm motility, sperm morphology, and DNA fragmentation) and other male patient parameters (e.g., changes in serum hormone concentrations (e.g., FSH, LH, inhibin B, testosterone, estradiol) and changes in free testosterone concentration) will be measured and analyzed. Semen and sperm parameters will be by assessed by laboratory methodologies known in the field, such as those in accordance with World Health Organisation (WHO) standards. See, e.g., Boitrelle et al., Life (2021). Additionally, subjects may assess their own sperm parameters using a sperm testing device, such as the ExSeed® device (ExSeed Health, Denmark), or other sperm testing device, such as those reviewed in Onofre, et al., Facts Views Vis ObGyn (2021) 13 (1): 79-93.

All cases of spontaneous pregnancy of the female partner occurring within 9 months after the male subject's date of randomisation will be followed for the duration of the pregnancy to collect information on pregnancy losses and live births. Furthermore, data will be collected on minor/major congenital anomalies of neonates, at birth, 4 weeks, and 1 year after birth.

### Study Outcomes

The primary endpoint of the study is spontaneous pregnancy observed in the female partners within 9 months after start of treatment of the male subjects, where spontaneous pregnancy is defined as vital pregnancy (documentation of at least one intrauterine gestational sac with fetal heartbeat by ultrasound). The secondary objectives are positive urine BhCG of the female partner, time to spontaneous pregnancy, changes in semen parameters (e.g., semen volume, sperm concentration, total sperm count, sperm motility, sperm morphology, and semen DNA fragmentation), changes in serum hormone concentrations (e.g., FSH, LH, inhibin B, testosterone, estradiol), and changes in free testosterone concentration, objectively measuring the effect of FE 999049 treatment on male subjects. Safety is assessed by evaluating blood samples for assessment of anti FSH antibodies and identifying immune-related adverse events.

### Results

The ADAM study will be the first study to use FE 999049 (follitropin delta) for the treatment of idiopathic infertility in a male patient. The present inventors believe a daily dose of 12 μg FE 999049, which is rFSH including α2,3-sialylation and α2,6-sialylation, will be safe and well-tolerated in the patient population described herein. Further, the present inventors believe the high daily dose of 12 μg FE 999049 (rFSH including α2,3-sialylation and α2,6-sialylation) will provide significantly greater (e.g., as measured by AUC and Cₘₐₓ) and more consistent FSH exposure than is observed with daily administration of conventional (e.g., urinary or CHO-cell derived) FSH.

The increased FSH dose compared to previous studies (see e.g. Ding and Cannarella mentioned at page 2 above) is anticipated to provide a better indication of the maximal possible treatment effect on spontaneous pregnancy rate and semen parameters. The trial population was carefully defined to include men with a diagnosis of unexplained infertility, including oligoasthenozoospermia, consistent with possible benefit from FSH treatment. The primary endpoint is spontaneous vital pregnancy observed in the female partner within 9 months after randomization of the male subject, and secondary endpoints include changes in semen parameters. A Bayesian framework for decision making in the presence of a single target treatment effect, which is defined such that efficacy above this target is of interest, will be used. The trial aims to demonstrate improvement in the spontaneous pregnancy rate of at least 10% when comparing FE 999049 and placebo treatment groups. The Proof-of-Concept conclusion will be based on the primary endpoint demonstrating evidence of statistical significance and clinical relevance using Bayesian decision criteria.

The inventors believe the therapeutic compositions and methods described herein will provide an improvement in spontaneous pregnancy rate of 10% when comparing the FE 999049 treatment group to the placebo treatment group; such an improvement is clinically significant. Accordingly, the present inventors believe the dosing regimen described herein will improve the chance of spontaneous pregnancy observed in female partners of the treated subjects in comparison to placebo (inactive treatment).

There have been disclosed hereinbefore the products, products for use, uses and methods (recombinant FSHs, recombinant FSH products, pharmaceutical compositions, uses and methods) defined by the following numbered paragraphs:

1. A composition comprising rFSH for use in the treatment of idiopathic infertility in a male patient, wherein the rFSH includes α2,3-sialylation and α2,6-sialylation, wherein the rFSH is for administration to the patient at a daily dose of 11-13 μg (e.g., 12 μg).

2. A composition comprising rFSH for use in the treatment of infertility in a male patient, wherein the rFSH includes α2,3-sialylation and α2,6-sialylation, wherein the rFSH is for administration to the patient at a daily dose of 11-13 μg (e.g., 12 μg).

3. A composition comprising rFSH for use in the treatment of male factor infertility in a patient, wherein the rFSH includes α2,3-sialylation and α2,6-sialylation, wherein the rFSH is for administration to the patient at a daily dose of 11-13 μg (e.g., 12 μg).

4. A composition for use according to paragraph 1, 2 or 3, wherein the infertility is at least one sperm-related infertility selected from azoospermia, oligospermia, asthenozoospermia, teratozoospermia, parvisemia, spermatocytopenia and malformation.

5. A composition for use according to any preceding paragraph, wherein from 5 to 20% of the total sialylation is α2,6-sialylation and/or wherein the rFSH is produced or expressed in a PERC6® cell line.

6. A composition for use according to any preceding paragraph, wherein the administration to the patient at a daily dose of 11-13 μg (e.g., 12 μg) is for a treatment period selected from (i) at least 28 days; (ii) from 28 days to 6 months; (iii) at least 30 days; (iv) from 30 days to 6 months; (v) 28 days; (vi) 30 days; (vii) 6 weeks; (viii) 8 weeks; (ix) 2 months; (x) 10 weeks; (xi) 12 weeks; (xii) 3 months; (xiii) 14 weeks; (xiv) 16 weeks; (xv) 4 months; (xvi) 18 weeks; (xvii) 20 weeks; (xviii) 5 months; (xix) 22 weeks, (xx) 24 weeks, and (xxi) 6 months.

7. A composition for use according to any preceding paragraph, wherein the patient has a Body Mass Index (BMI)≤35 kg/m².

8. A composition for use according to any preceding paragraph, wherein the patient has a semen volume ≥1.4 mL.

9. A composition for use according to any preceding paragraph, wherein the patient has a baseline total sperm count of 5 to 39 million.

10. A composition for use according to any preceding paragraph, wherein the patient has a sperm concentration less than 5 million spermatozoa per mL.

11. A composition for use according to any preceding paragraph, wherein the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL.

12. A composition for use according to any preceding paragraph, wherein the patient has a total motile sperm count of 5 to 16 million.

13. A composition for use according to any preceding paragraph, wherein the patient has ≥15% spermatozoa with normal morphology (an oval-shaped head, an intact midpiece and an uncoiled single tail and ability to swim well and in a straight line).

14. A composition for use according to any preceding paragraph, wherein the patient has a baseline serum hormone concentration of follicle-stimulating hormone (FSH) of 1.5 to 8.0 IU/L.

15. A composition for use according to any preceding paragraph, wherein the patient has a baseline serum hormone concentration of luteinising hormone (LH) of 1.2 to 7.5 IU/L.

16. A composition for use according to any preceding paragraph, wherein the patient has a baseline serum hormone concentration of testosterone of ≥300 ng/dl (equals ≥10.4 nmol/L).

17. A composition comprising rFSH for use in the treatment of testosterone deficiency in a male patient, wherein the rFSH includes α2,3-sialylation and α2,6-sialylation, and wherein the rFSH is for administration to the patient at a daily dose of 11-13 μg (e.g., 12 μg).

18. A composition comprising rFSH for use in the treatment of idiopathic oligospermia in a male patient, wherein the rFSH includes α2,3-sialylation and α2,6-sialylation, and wherein the rFSH is for administration to the patient at a daily dose of 11-13 μg (e.g., 12 μg).

19. A method of treating infertility in a male patient, comprising;
administering to the patient rFSH including α2,3-sialylation and α2,6-sialylation, wherein the rFSH is administered at a dose of 11-13 μg per day, optionally at a dose of 12 μg per day.

20 The method of paragraph 19, wherein the infertility is male factor infertility. 21 The method of paragraph 19, wherein the infertility is idiopathic infertility.

22. The method of paragraph 19, wherein the infertility is at least one sperm-related infertility selected from azoospermia, oligospermia, asthenozoospermia, teratozoospermia, parvisemia, spermatocytopenia and malformation.

23. A method of treating testosterone deficiency in a male patient, comprising administering to the patient rFSH including α2,3-sialylation and α2,6-sialylation, wherein the rFSH is administered at a dose of 11-13 μg per day, optionally at a dose of 12 μg per day.

24. A method of treating idiopathic oligospermia in a male patient, comprising administering to the patient rFSH including α2,3-sialylation and α2,6-sialylation, wherein the rFSH is administered at a dose of 11-13 μg per day, optionally at a dose of 12 μg per day.

25. A method of any one of paragraphs 19-24, wherein from 5 to 20% of the total sialylation of the rFSH is α2,6-sialylation.

26. A method of any one of paragraphs 19-25, wherein the rFSH is produced or expressed in a PERC6® cell line.

27. A method of any one of paragraphs 19-26, wherein the method comprises administering the rFSH to the patient at a daily dose of 11-13 μg for a treatment period selected from (i) at least 28 days; (ii) from 28 days to 6 months; (iii) at least 30 days; (iv) from 30 days to 6 months; (v) 28 days; (vi) 30 days; (vii) 6 weeks; (viii) 8 weeks; (ix) 2 months; (x) 10 weeks; (xi) 12 weeks; (xii) 3 months; (xiii) 14 weeks; (xiv) 16 weeks; (xv) 4 months; (xvi) 18 weeks; (xvii) 20 weeks; (xviii) 5 months; (xix) 22 weeks, (xx) 24 weeks, and (xxi) 6 months.

28. A method of any one of paragraphs 19-27, wherein the patient has one or more or all of the following characteristics prior to treatment:
a Body Mass Index (BMI)≤35 kg/m²;
a semen volume ≥1.4 mL;
a total sperm count of 5 to 39 million;
a sperm concentration less than 5 million spermatozoa per mL;
a sperm concentration less than or equal to 3 million spermatozoa per mL;
a total motile sperm count of 5 to 16 million;
≥15% spermatozoa with normal morphology;
a serum concentration of follicle-stimulating hormone (FSH) of 1.5 to 8.0 IU/L;
a serum concentration of luteinising hormone (LH) of 1.2 to 7.5 IU/L;
a serum concentration of testosterone of ≥300 ng/dl (≥10.4 nmol/L).

29. A method of any one of paragraphs 19-28, wherein the patient has a sperm concentration selected from less than 5 million spermatozoa per mL, less than 4 million spermatozoa per mL, less than or equal to 3 million spermatozoa per mL, less than or equal to 2 million spermatozoa per mL, less than or equal to 1 million sperm per mL, and less than or equal to 0.5 million sperm per mL.

30. A method of any one of paragraphs 19-29, wherein the method is effective to provide an improved chance of spontaneous pregnancy in a female partner of the treated male patient.

31. A method of any one of paragraphs 19-30, wherein the method is effective to improve one or more or all of semen volume, sperm concentration, total sperm count, sperm motility, and sperm morphology, or is effective to reduce semen DNA fragmentation, of sperm/semen of the treated male patient.

32. A method of any one of paragraphs 19-31, wherein the method is effective to improve the chance of a positive beta-human chorionic gonadotropins (BhCG) urine test in a female partner of the treated subject in comparison to men treated with placebo.

33. A method of any one of paragraphs 19-32, wherein the method is effective to reduce the time to spontaneous pregnancy in a female partner of the treated subject in comparison to men treated with placebo.

1. A composition comprising rFSH for use in the treatment of idiopathic infertility in a male patient, wherein the rFSH includes α2,3-sialylation and α2,6-sialylation, wherein the rFSH is for administration to the patient at a daily dose of 12-13 μg (e.g., 12 μg).

2. A composition comprising rFSH for use in the treatment of infertility in a male patient, wherein the rFSH includes α2,3-sialylation and α2,6-sialylation, wherein the rFSH is for administration to the patient at a daily dose of 12-13 μg (e.g., 12 μg).

3. A composition comprising rFSH for use in the treatment of male factor infertility in a patient, wherein the rFSH includes α2,3-sialylation and α2,6-sialylation, wherein the rFSH is for administration to the patient at a daily dose of 12-13 μg (e.g., 12 μg).

4. A composition for use according to claim 1, 2 or 3, wherein the infertility is at least one sperm-related infertility selected from azoospermia, oligospermia, asthenozoospermia, teratozoospermia, parvisemia, spermatocytopenia and malformation.

5. A composition for use according to any preceding claim, wherein from 5 to 20% of the total sialylation is α2,6-sialylation and/or wherein the rFSH is produced or expressed in a PERC6® cell line.

6. A composition for use according to any preceding claim, wherein the administration to the patient at a daily dose of 12-13 μg (e.g., 12 μg) is for a treatment period selected from (i) at least 28 days; (ii) from 28 days to 6 months; (iii) at least 30 days; (iv) from 30 days to 6 months; (v) 28 days; (vi) 30 days; (vii) 6 weeks; (viii) 8 weeks; (ix) 2 months; (x) 10 weeks; (xi) 12 weeks; (xii) 3 months; (xiii) 14 weeks; (xiv) 16 weeks; (xv) 4 months; (xvi) 18 weeks; (xvii) 20 weeks; (xviii) 5 months; (xix) 22 weeks, (xx) 24 weeks, and (xxi) 6 months.

7. A composition for use according to any preceding claim, wherein the patient has a Body Mass Index (BMI)≤35 kg/m².

8. A composition for use according to any preceding claim, wherein the patient has a semen volume ≥1.4 mL.

9. A composition for use according to any preceding claim, wherein the patient has a baseline total sperm count of 5 to 39 million.

10. A composition for use according to any preceding claim, wherein the patient has a sperm concentration less than 5 million spermatozoa per mL.

11. A composition for use according to any preceding claim, wherein the patient has a sperm concentration less than or equal to 3 million spermatozoa per mL, for example less than or equal to 2 million spermatozoa per mL, for example less than or equal to 1 million sperm per mL, for example less than or equal to 0.5 million sperm per mL.

12. A composition for use according to any preceding claim, wherein the patient has a total motile sperm count of 5 to 16 million.

13. A composition for use according to any preceding claim, wherein the patient has ≥15% spermatozoa with normal morphology (an oval-shaped head, an intact midpiece and an uncoiled single tail and ability to swim well and in a straight line), and/or wherein the patient has a baseline serum hormone concentration of follicle-stimulating hormone (FSH) of 1.5 to 8.0 IU/L, and/or wherein the patient has a baseline serum hormone concentration of luteinising hormone (LH) of 1.2 to 7.5 IU/L, and/or wherein the patient has a baseline serum hormone concentration of testosterone of ≥300 ng/dl (equals ≥10.4 nmol/L).

14. A composition comprising rFSH for use in the treatment of testosterone deficiency in a male patient, wherein the rFSH includes α2,3-sialylation and α2,6-sialylation, and wherein the rFSH is for administration to the patient at a daily dose of 11-13 μg (e.g., 12 μg).

15. A composition comprising rFSH for use in the treatment of idiopathic oligospermia in a male patient, wherein the rFSH includes α2,3-sialylation and α2,6-sialylation, and wherein the rFSH is for administration to the patient at a daily dose of 12-13 μg (e.g., 12 μg).
